(19) United States
(12) Patent Application Publication
(10) Pub. No.: US 2021/0232768 A1
(43) Pub. Date: Jul. 29, 2021

(54) MACHINE LEARNING MODEL WITH EVOLVING DOMAIN-SPECIFIC LEXICON FEATURES FOR TEXT ANNOTATION

(71) Applicant: KONINKLIJKE PHILIPS N.V., ElNDHOVEN (NL)

(72) Inventors: Yuan Ling, Somerville, MA (US); Sheikh Sadid Al Hasan, Cambridge, MA (US); Oladimeji Feyisetan Farri, Yorktown Heights, NY (US); Junyi Liu, Windham, NH (US)

(21) Appl. No.: 17/048,708

(22) PCT Filed: Apr. 18, 2019
(86) PCT No.: PCT/EP2019/060212

§ 371 Date: Oct. 19, 2020

Related U.S. Application Data
(60) Provisional application No. 62/659,998, filed on Apr. 19, 2018.

(51) Int. Cl. G06F 40/295; G06N 3/04; G06N 20/00; G06F 16/901

(52) U.S. Cl. CPC G06F 40/295; G06F 16/9027; G06N 20/00; G06N 3/0454

(57) ABSTRACT

A method of generating embeddings for a machine learning model, including: extracting a character embedding and a word embedding from a first textual data; generating a domain knowledge embedding from a domain knowledge dataset; combining the character embedding, the word embedding, and the domain knowledge embedding into a combined embedding; and providing the combined embedding to a layer of the machine learning model.

### TECHNICAL FIELD

Various exemplary embodiments disclosed herein relate generally to a machine learning model with evolving domain-specific lexicon features for natural language processing.

### BACKGROUND

Machine learning models may be developed to annotate named entities in text, e.g., identifying the names of individuals or places, dates, animals, diseases, etc. In the biomedical setting, disorder annotation is a feature in many biomedical natural language processing applications. For example, extracting disorder names from clinical trials text may be helpful for patient profiling and other downstream applications such as matching clinical trials to eligible patients. Similarly, disorder annotation in biomedical articles can help information search engines to accurately index them such that clinicians can easily find relevant articles to enhance their knowledge.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method of generating embeddings for a machine learning model, including extracting a character embedding and a word embedding from a first textual data; generating a domain knowledge embedding from a domain knowledge dataset; combining the character embedding, the word embedding, and the domain knowledge embedding into a combined embedding; and providing the combined embedding to a layer of the machine learning model.

Various embodiments are described, wherein the domain knowledge dataset includes feedback from a domain expert.

Various embodiments are described, wherein the feedback from the domain expert includes named entity recognition labeling of a second textual data.

Various embodiments are described, wherein the feedback from the domain expert includes additional vocabulary to be used to update a vocabulary database.

Various embodiments are described, wherein the feedback from the domain expert is based upon a determination of the correctness of the output of the machine learning model.

Various embodiments are described, wherein the domain knowledge dataset includes the output of a natural language processing engine applied to a second textual data.

Various embodiments are described, wherein the domain knowledge dataset includes the output of a query based upon a second textual data to a TRIE dictionary based upon a vocabulary data.

Various embodiments are described, wherein the machine learning model performs named entity recognition of a second textual data.

Various embodiments are described, wherein the machine learning model performs medical disorder annotation of a second textual data.

Various embodiments are described, further including: training the machine learning model using the first textual data, the character embedding, and the word embedding before generating the domain knowledge embedding; and retraining the machine learning model after generating the domain knowledge embedding.

Various embodiments are described, further including: determining that retraining of the machine learning model is required based upon the amount of data added to the domain knowledge dataset before retraining the machine learning model.

Various embodiments are described, wherein extracting the character embedding further includes: applying a convolutional neural network layer to words in the first textual data to produce a first character embedding portion; applying a long short term memory neural network layer to words in the first textual data to produce a second character embedding portion; and concatenating the first character embedding portion and the second character embedding portion to produce the character embedding.

Various embodiments are described, wherein the machine learning model includes a long short term memory layer and a conditional random field layer and further includes providing the domain knowledge embedding to the conditional random field layer.

Various embodiments are described, further including: training the machine learning model using the first textual data, the character embedding and the word embedding before generating the domain knowledge embedding; and retraining the machine learning model after generating the domain knowledge embedding.

Further various embodiments relate to a non-transitory machine-readable storage medium encoded with instructions for generating embeddings for a machine learning model, including: instructions for extracting a character embedding and a word embedding from a first textual data; instructions for generating a domain knowledge embedding from a domain knowledge dataset; instructions for combining the character embedding, the word embedding, and the domain knowledge embedding into a combined embedding; and instructions for providing the combined embedding to a layer of the machine learning model.

Various embodiments are described, wherein the domain knowledge dataset includes feedback from a domain expert.

Various embodiments are described, wherein the feedback from the domain expert includes named entity recognition labeling of a second textual data.

Various embodiments are described, wherein the feedback from the domain expert includes additional vocabulary to be used to update a vocabulary database.

Various embodiments are described, wherein the feedback from the domain expert is based upon a determination of the correctness of the output of the machine learning model.

Various embodiments are described, wherein the domain knowledge dataset includes the output of a natural language processing engine applied to a second textual data.

Various embodiments are described, wherein the domain knowledge dataset includes the output of a query based upon a second textual data to a TRIE dictionary based upon a vocabulary data.

Various embodiments are described, wherein the machine learning model performs named entity recognition of a second textual data.

Various embodiments are described, wherein the machine learning model performs medical disorder annotation of a second textual data.

Various embodiments are described, further including: instructions for training the machine learning model using the first textual data, the character embedding, and the word embedding before generating the domain knowledge embedding; and instructions for retraining the machine learning model after generating the domain knowledge embedding.

Various embodiments are described, further including: instructions for determining that retraining of the machine learning model is required based upon the amount of data added to the domain knowledge dataset before retraining the machine learning model.

Various embodiments are described, wherein extracting the character embedding further includes: instructions for applying a convolutional neural network layer to words in the first textual data to produce a first character embedding portion; instructions for applying a long short term memory neural network layer to words in the first textual data to produce a second character embedding portion; and instructions for concatenating the first character embedding portion and the second character embedding portion to produce the character embedding.

Various embodiments are described, wherein the machine learning model includes a long short term memory layer and a conditional random field layer and further includes instructions for providing the domain knowledge embedding to the conditional random field layer.

Various embodiments are described, further including: instructions for training the machine learning model using the first textual data, the character embedding and the word embedding before generating the domain knowledge embedding; and instructions for retraining the machine learning model after generating the domain knowledge embedding.

Further various embodiments relate to a non-transitory machine-readable storage medium encoded with instructions for generating embeddings for a disorder annotation machine learning model, including: instructions for extracting a character embedding and a word embedding from a first textual data; instructions for generating a lexicon embedding from a lexicon dataset; instructions for generating an extra tagging embedding from an extra tagging dataset; instructions for combining the character embedding, the word embedding, the lexicon embedding, and extra tagging embedding into a combined embedding; and instructions for providing the combined embedding to a layer of the disorder annotation machine learning model.

Various embodiments are described, wherein the extra tagging dataset includes feedback from a domain expert.

Various embodiments are described, wherein the feedback from the domain expert includes disorder annotation of a second textual data.

Various embodiments are described, wherein the feedback from the domain expert includes additional vocabulary to be used to update a vocabulary database.

Various embodiments are described, wherein the feedback from the domain expert is based upon a determination of the correctness of the output of the disorder annotation machine learning model.

Various embodiments are described, wherein the lexicon dataset includes the output of a natural language processing engine applied to a second textual data.

Various embodiments are described, wherein the lexicon dataset includes the output of a query based upon a second textual data to a TRIE dictionary based upon a vocabulary data.

Various embodiments are described, further including: instructions for training the disorder annotation machine learning model using the first textual data, the character embedding, and the word embedding before generating the lexicon embedding and the extra tagging embedding; and instructions for retraining the disorder annotation machine learning model after generating the lexicon embedding and the extra tagging embedding.

Various embodiments are described, further including: instructions for determining that retraining of the disorder annotation machine learning model is required based upon the amount of data added to the lexicon dataset and extra tagging dataset before retraining the disorder annotation machine learning model.

Various embodiments are described, wherein extracting the character embedding further includes: instructions for applying a convolutional neural network layer to words in the first textual data to produce a first character embedding portion; instructions for applying a long short term memory neural network layer to words in the first textual data to produce a second character embedding portion; and instructions for concatenating the first character embedding portion and the second character embedding portion to produce the character embedding.

Various embodiments are described, wherein the disorder annotation machine learning model includes a long short term memory layer and a conditional random field layer and further includes instructions for providing the lexicon embedding and the extra tagging embedding to the conditional random field layer.

### BRIEF DESCRIPTION OF THE DRAWINGS

In order to better understand various exemplary embodiments, reference is made to the accompanying drawings, wherein:

FIG. 1 illustrates an architecture of LSTM-CRF model for disorder annotation;

FIG. 2 illustrates how lexicon embedding and extra tagging embedding may be generated;

FIG. 3 illustrates a disorder annotation system using extra tagging embedding and lexicon embedding; and

FIG. 4 illustrates an LSTM-CRF model that is trained in a first domain that may be migrated for use in a second domain.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Disorder annotation is important in many biomedical natural language applications. For example, extracting disorder names from clinical trials text may be helpful for patient profiling and other downstream applications such as matching clinical trials to eligible patients. Similarly, disorder annotation in biomedical articles can help information search engines to accurately index them such that clinicians can easily find relevant articles to enhance their knowledge. Achieving high precision and high recall in disorder annotation is desired by most real-world applications.

Deep learning techniques have demonstrated superior performance over traditional machine learning (ML) techniques for various general-domain natural language processing (NLP) tasks e.g., language modeling, parts-of-speech (POS) tagging, named entity recognition (NER), paraphrase identification, sentiment analysis etc. Clinical documents pose unique challenges compared to general-domain text due to widespread use of acronyms and non-standard clinical jargons by healthcare providers, inconsistent document structure and organization, and a requirement for rigorous de-identification and anonymization to ensure patient data privacy. These methods also depend on well-labeled datasets, and as a result, the models need to be re-trained every time when applied to a new dataset. Further, in some situations, there is not enough labeled data for training the model. Overcoming these challenges could foster more research and innovation for various useful clinical applications including clinical decision support, patient cohort identification, patient engagement support, population health management, pharmacovigilance, personalized medicine, and clinical text summarization.

To this end, embodiments will be described that address the disorder annotation task by encoding clinical domain knowledge via various types of embeddings into different layers of a deep neural network architecture including a long short-term memory network-conditional random field (LSTM-CRF) model and convolutional neural network (CNN) model. Experiments using these embodiments show the impact of clinical domain knowledge on the performance of the model while adding this clinical domain knowledge at different parts of the network. These embodiments also achieve new state-of-the-art results in disorder annotation on a scientific article dataset.

Embodiments will be described herein that illustrate the training of a model on a well-labeled dataset while being capable to apply the trained model to a new unlabeled dataset without losing important domain-specific features for the new dataset. These embodiments train a LSTM-CRF model for disorder annotation based on well-labeled scientific article text data. The LSTM-CRF model further encodes domain-specific lexicon features from a general dictionary. Additionally, the LSTM-CRF model encodes evolving feedback from the unlabeled corpus. Thus, even though the LSTM-CRF model is trained on one specific dataset, the LSTM-CRF model may be applied to a different dataset with evolving lexicon features. Details of these features will be further described below. The embodiments described below are related to disorder recognition in the biomedical field, where the size of labeled data sets may be small, but the data sets to be analyzed are large. This situation arises in other areas as well, and hence the embodiments described herein can be widely applied, such as where a model is trained on one set of data in a first domain, and that model is then expanded and applied to data in a second domain.

Disorder annotation from free text is a sequence tagging problem. A BIO tagging schema can be used for tagging the input sequence. For example, as shown below, the tagging results denote a tag for each word from input text. “B-disorder” represents the beginning word of a disorder name, “I-disorder” represents the other word in a disorder name, and “O” represents a word not belonging to a disorder name:
Input Text: . . . new diagnoses of prostate cancer . . .
Tagging Results: O O O B-disorder I-disorder.

Existing rule-based systems or traditional machine learning methods for disorder annotation heavily depend on hand-crafted features, such as syntactic, lexical, N-gram, etc. Neural network based methods usually don't rely on hand-crafted features, however, a large labelled data is required to train the neural network. In the embodiments described herein, domain knowledge is introduced into the neural network based method.

For disorder annotation, there are many existing clinical NLP engines that may be used. It would be good to take advantage of existing tools instead of training a neural network based model from scratch purely on a labelled dataset, which may be limited. Thus, the embodiments described herein encode output from existing clinical NLP pipelines to improve the model performance for disorder annotation.

A hybrid clinical NLP engine may be used to generate tagging output, but any other type of clinical NLP pipeline may be used for this purpose. The clinical NLP engine generates disorder tagging and other types of biomedical concepts. In the embodiments described below, only the disorder tagging is used, but the other types of tagging may also provide useful information that may be encoded in the model as well.

Another type of domain knowledge is disease vocabulary. Prior research spent significant effort to build dictionaries/ontologies to facilitate biomedical NLP tasks. MEDIC is an example of an existing disease vocabulary, which includes 9,700 unique diseases and 67,000 unique terms in total.

Outputs from clinical NLP engines and disease vocabulary are two kinds of domain knowledge used by the embodiments described herein to improve the neural network based method for disorder annotation. Other sorts of or domain information may be identified and used to improve performance of neural networks as described by embodiments disclosed herein. This additional domain information allows for the improvement in performance of neural network based methods for annotation and other tasks when the data labeled data sets are small or when moving a model from one domain to another.

As described above, the LSTM-CRF model has been developed to perform NER, and the LSTM-CRF model achieves state-of-the-art performance in the general domain. Thus, this model may be adopted to the task of disorder annotation. However, in a real use case, currently there is not enough labeled data for training a model to extract disorder names from clinical trials text. The only available dataset is scientific articles with disorder names annotated. As a result, the following issues may be considered in determining how to apply a LSTM-CRF model to the problem of disorder annotation: first, how to adapt the LSTM-CRF model trained on one corpus to another new corpus; second, how to encode lexicon features from the new corpus, and third, how to efficiently encode and update the feedback from domain experts into the trained model. The embodiments described herein address these various issues.

Embodiments of a LSTM-CRF model for disorder annotation will now be described. The generic architecture of neural network for named entity recognition task is a bidirectional LSTM-CRF that takes as input a sequence of vectors (x₁, x₂, . . . , xₙ) and returns another sequence (y₁, y₂, . . . , yₙ) that represents tagging information of the input sequence correspondingly.

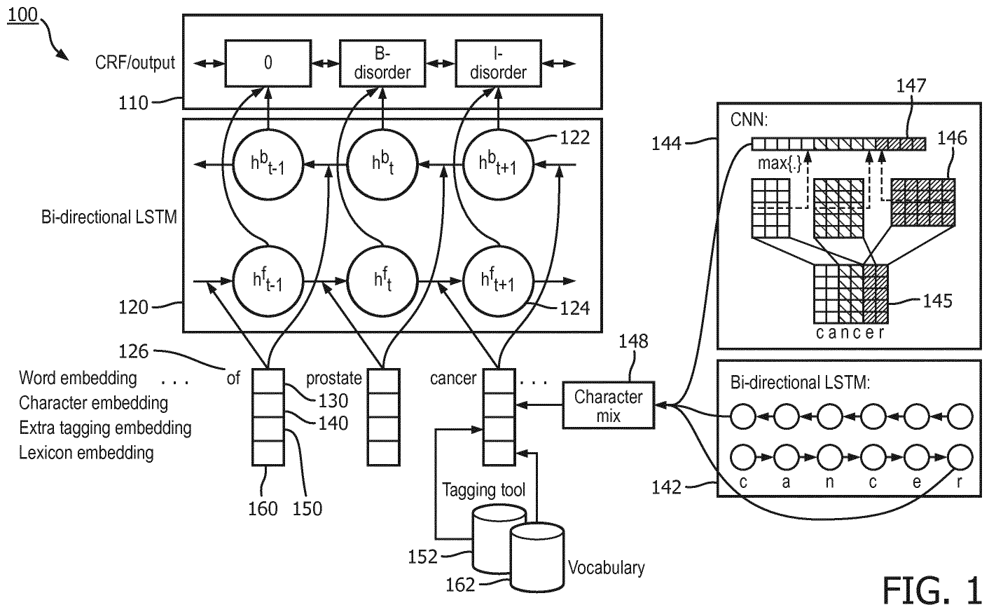

FIG. 1 illustrates an architecture of LSTM-CRF model for disorder annotation. The LSTM-CRF model 100 includes the following layers: a character embedding layer 140, a word embedding layer 130, a bi-directional LSTM layer 120, a CRF tagging layer 110. For a given sentence (x₁, x₂, . . . , xₙ) containing n words, each word is represented as a d-dimensional vector. The d-dimensional vector is concatenated from two parts: a d1-dimensional vector V_{char} from the character embedding layer 140 and a d2-dimensional vector V_{word} from the word embedding layer 130. The bi-directional LSTM layer 120 reads the vector representations of input sentence (x₁, x₂, . . . , xₙ) to produce two sequences of hidden vectors, i.e., a forward sequence (h₁^{f}, h₂^{f}, . . . hₙ^{f}) 124 and a backward sequence (h₁^{b}, h₂^{b}, . . . , hₙ^{b}) 122. The LSTM layer 120 then concatenates the forward sequence 124 and the backward sequence 122 into hᵢ=[hᵢ^{f}; hᵢ^{b}], which is then input into the CRF layer 110. The CRF layer 110 then determines and outputs the label yᵢ for the specific input word xᵢ.

The encoding of the character embedding layer 140 may be accomplished using various methods. Two possible methods include using a character bi-directional LSTM layer 142 for learning character embedding and a character convolutional neural network (CNN) layer 144 for learning character embedding. The bi-directional LSTM layer 142 provides embedded information, among other information, related to the sequence of letters in the words received, for example, Greek or Latin cognates. The CNN layer 144 provides embedded information, among other information, relative to which letters in a word are the most useful in determining the meaning of the word.

The character CNN layer 144 generates a character embedding for each word in sentence as follows. First, a vocabulary of characters C is defined. Let d be the dimensionality of character embeddings, and Q∈R^{d×|C|} is the matrix character embeddings. The character CNN layer 144 takes the current word “cancer” as input and performs a lookup of Q∈R^{d×|C|} and stacks the lookup results to form the matrix C^{k} 145. The convolution operations are applied between C^{k} 145 and multiple filter/kernel matrices 147. Then a max-over-time pooling operation is applied to obtain a fixed-dimensional representation of the word, which is denoted as V_{cnn} 147. This specific CNN layer 144 is intended to be an example, and other CNN or recurrent neural network (RNN) layers with various operations and number of layers may also be used.

The character LSTM layer 142 is similar to the bi-directional LSTM layer 120 in the architecture of LSTM-CRF model 100. Instead of taking a sequence of words in sentence as input as is done in the LSTM layer 120, the character LSTM layer 142 takes a sequence of characters in a word as input. The character LSTM layer 142 then outputs the concatenate the final step of two sequences [hₜ^{f}; hₜ^{b}], which may be denoted it as Vₗₛₜₘ.

Both the character CNN layer 144 and the character LSTM layer 142 are used to learn the character embeddings. A character MIX layer 148 takes the outputs from both the character CNN layer 144 and the character LSTM layer 142 and concatenates them into Vₘᵢₓ=[V_{cnn}; Vₗₛₜₘ], which is the same d1-dimensional vector V_{char} for the character embedding layer 140 that is discussed above.

In the LSTM-CRF model 100, domain knowledge either from domain vocabulary 162 or external tagging tools 152 may be introduced through a lexicon embedding layer 150 and an extra tagging embedding layer 160.

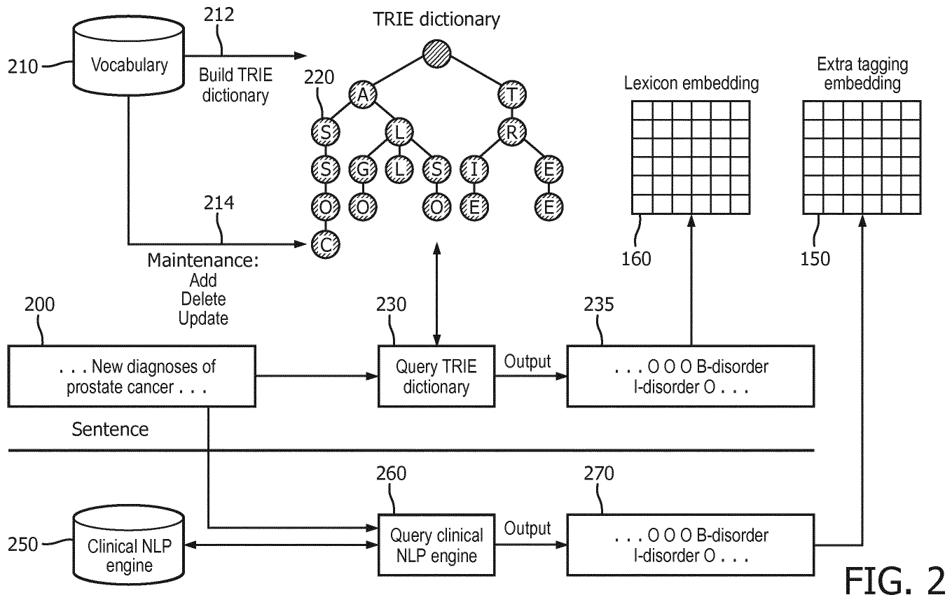

FIG. 2 illustrates how lexicon embedding and extra tagging embedding may be generated.

Prior knowledge existing in vocabulary plays an important role in biomedical NLP tasks. Lots of rule-based systems or traditional machine learning systems based on hand-crafted features have been developed, which utilize vocabulary to obtain prior domain knowledge, especially in biomedical the NLP domain. The integration of this domain knowledge can be helpful in entity recognition tasks.

Generating the lexicon embedding utilizes a vocabulary database 210. The vocabulary database 210 is used to build 212 a TRIE dictionary 220 for the vocabulary. The TRIE dictionary 220 may easily be maintained 214 as well by updating the TRIE dictionary 220 when new entries are added to, entries are deleted from, or entries are updated in the vocabulary database 210. The TRIE is an efficient data structure for frequent words/phrases matching. An input sentence 200 is received and the TRIE dictionary 220 is queried 230. Based on any matching results, the query provides a tagging sequence as output. For example, in the sentence “ . . . new diagnoses of prostate cancer . . . ”, the phrase “prostate cancer” is mapped in TRIE dictionary, so the query will tag the phrase “prostate cancer” as “B-disorder I-disorder”. The tagging results 235 are further used to generate the lexicon embedding Vₗₑₓ 160. This is accomplished by creating an entry for the tagged phrase, “prostate cancer” in this example, in the lexicon embedding matrix 160. The embedded values associated with the new entry may be randomized to improve the convergence of the embedded values during the LSTM-CRF model training.

The generating of extra tagging embedding is similar to generating the lexicon embedding as discussed above. Generating the extra tagging embedding may utilize a clinical NLP engine 250 instead of using a vocabulary database. For each input sentence 200, the clinical NLP engine 250 is queried 260, and the tagging sequence is output. The tagging results 270 are further used to generate the extra tagging embedding V_{tag} 150. This is accomplished by creating an entry for the tagged phrase, “prostate cancer” in this example, in the extra tagging embedding matrix 150. The embedded values associated with the new entry may be randomized to improve the convergence of the embedded values during the LSTM-CRF model training.

The lexicon embedding 160 and the extra tagging embedding 150 may also be updated using other methods. One method could involve human domain experts who identify disorders in unlabeled text or who analyze the output of the LSTM-CRF model 100 to identify errors, and such feedback may be used to update the lexicon embedding 160 or the extra tagging embedding 150. The input sentences 200 may come from an unlabeled corpus of interest.

The lexicon embedding Vₗₑₓ 160 and the extra tagging embedding V_{tag} 150 may be embedded into the architecture of LSTM-CRF model 100 as shown in FIG. 1. Specifically, the lexicon embedding Vₗₑₓ 160 and the extra tagging embedding V_{tag} 150 may be embedded before the bi-directional LSTM layer 120 by concatenating them with word embedding 130 and character embedding 140, which results in a concatenated vector [V_{word}; V_{char}; Vₗₑₓ; V_{tag}] and acts as an input for the bi-directional LSTM layer 120. These additional embedding may extend the capability and performance of the LSTM-CRF model 100 beyond what is possible using just the available well-labeled corpus for training. The lexicon embedding 160 and the extra tagging embedding 150, individually or in combination, may be called domain knowledge embedding. Domain knowledge embedding includes any embedding added to the LSTM-CRF model based upon domain knowledge.

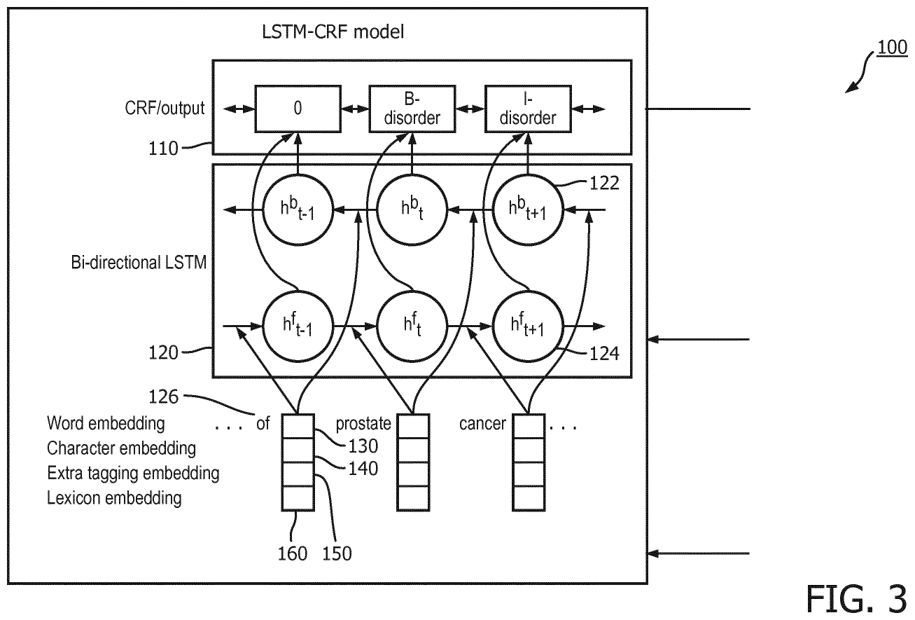

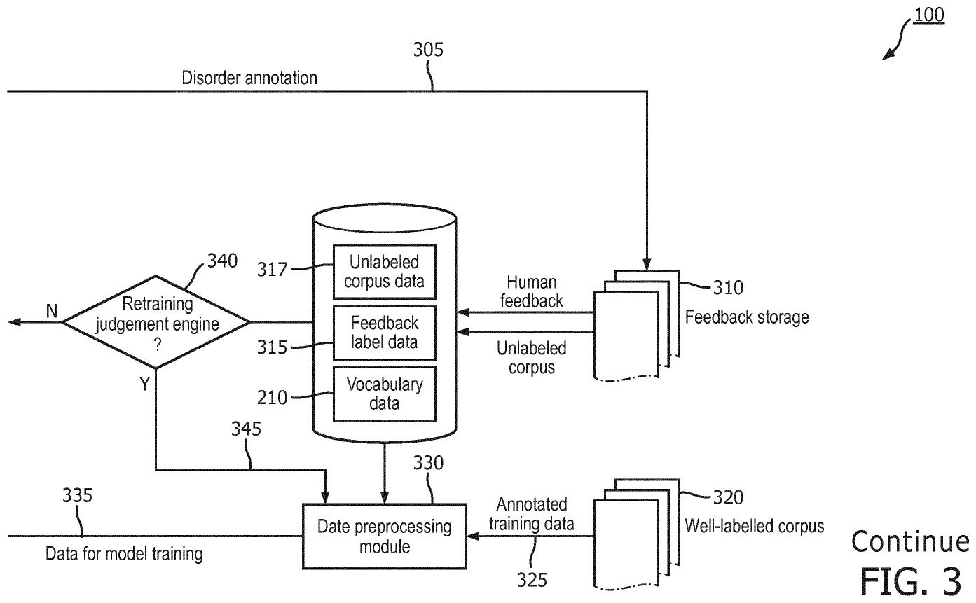

FIG. 3 illustrates a disorder annotation system using extra tagging embedding and lexicon embedding. The LSTM-CRF model 100 is the same as that described in FIG. 1. Initially, annotated training data 325 is extracted from a well-labelled corpus 320. A data preprocessing module 330 receives the annotated training data 325 and preprocesses this data to generate the initial word embedding data 130 and the character embedding data 120. Then the LSTM-CRF model 100 is trained using the training data 335. Then the LSTM-CRF model 100 may be deployed.

During deployment, the LSTM-CRF model may receive unlabeled data 126 and produce disorder annotations 305. These disorder annotations 305 may be stored in feedback storage 310 for analysis by a human domain expert. For example, the human domain expert may determine if the domain output annotations 305 output by the LSTM-CRF model are correct. Additionally, an unlabeled corpus may also be stored in feedback storage 310 for analysis by a human domain expert. The human domain expert may generate human feedback 311 that is stored in feedback label data storage 315. The human feedback may also be used to update the vocabulary data storage 210. Additionally, the unlabeled corpus 312 may be stored in the unlabeled corpus data storage 317.

A retraining judgement engine 340 may evaluate the updates to the feedback label storage, vocabulary label storage, and the unlabeled corpus storage to determine that sufficient additional amount of domain information has been received to justify retraining the LSTM-CRF model 100. This may be done by using various thresholds and metrics, for example, track the number of additions to the vocabulary storage 210 or feedback label storage 315. This decision may also consider the availability and cost of current processing assets that would be required to perform the retraining. Additionally, performance of the disorder annotation system may be monitored, and if the performance decreases below a specified threshold retraining may also be initiated. If retraining is not yet justified, the LSTM-CRF model 100 continues to operate. Once the retraining judgement engine 340 determines that retraining is needed, then such retraining request 345 is sent to the data preprocessing module 330.

When the data preprocessing module 330 receives a retraining request 345, it may create the extra tagging embedding data 150 and the lexicon embedding data 160 as described in FIG. 2 using an unlabeled corpus data as input. Further, the human feedback may be incorporated into one or both of the extra tagging embedding data 150 and the lexicon embedding data 160. Then the LSTM-CRF model 100 is retrained using the various updated data.

This retraining results in an updated and improved disorder annotation system and process. Over time as additional domain expert input is received along with additional vocabulary data and outputs from clinical NLP engines, the LSTM-CRF model improves the accuracy and scope of the disorder annotation process. Therefore, when only a small well-labeled corpus exists, the disorder annotation process may still be improved over time with the input of addition data from various sources using the extra tagging embedding and lexicon embedding. Again, as discussed above, these embodiments may be applied in other applications where all different sorts of domain knowledge may be gathered and input in to additional embedding layers that will improve the performance of an annotation process or other NLP processes. Examples of other annotation tasks or applications include parts-of-speech tagging, named entity recognition, event identification, semantic role labeling, temporal annotation, etc. where domain-specific vocabulary, terminology, ontology, corpora, etc. may provide additional knowledge to improve the performance of an annotation model.

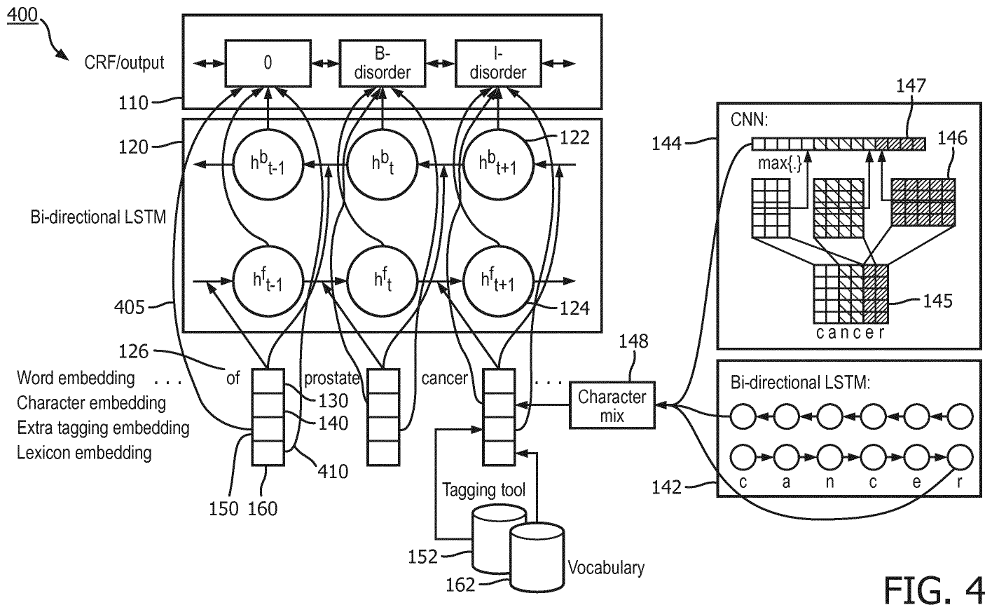

FIG. 4 illustrates an LSTM-CRF model that is trained in a first domain that may be migrated for use in a second domain. There are situations where a model developed in a first domain may be adapted for use in a second domain while retaining important domain specific features from the first domain. The LSTM-CRF model 400 is very similar to the LSTM-CRF model of FIG. 1. The LSTM-CRF model 400 retains the same labels from the LSTM-CRF model 100 of FIG. 1. The tagging tools 152 and the vocabulary tools 162 are used to generate domain specific knowledge as described above with respect to FIGS. 1 and 2. This domain specific knowledge is incorporated in the extra tagging embedding layer 150 and the lexicon embedding layer 160 as described above. The difference is that the information from extra tagging embedding layer 150 and the lexicon embedding layer 160 are also provided as inputs to the CRF layer 110. This is illustrated as a data connection 405 from the extra tagging embedding layer 150 to the CRF layer 110 and a data connection 410 from the lexicon embedding layer 160 to the CRF layer 110, which results a concatenated vector of [hᵢ^{f}; hᵢ^{b}; Vₗₑₓ; V_{tag}] as the input for the CRF layer 110. These additional connections 405 and 410 allow the additional domain knowledge encoded in the extra tagging embedding layer 150 and the lexicon embedding layer 160 to more directly affect the output of the LSTM-CRF model 400 at various layers of the architecture. This is accomplished by generating the data for extra tagging embedding layer 150 and the lexicon embedding layer 160 and then training the LSTM-CRF model with data from the second domain. As a result, valuable learning from the first domain may be retained while extending the model into a second domain.

Various features of the embodiments described above result in a technological improvement and advancement over existing disorder annotation systems, NER systems, and other NLP systems. Such features include, but are not limited to: the addition of lexicon embedding and extra tagging embedding based upon additional domain knowledge; extracting disorder information from an unlabeled corpus using clinical NLP engines, vocabulary databases implemented as a TRIE dictionary, and feedback information from domain experts; the use of CNN layers along with LSTM layers on the characters of a word; and using the lexicon embedding and extra tagging embedding information as an input to the CRF layer.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, or other similar devices.

The memory may include various memories such as, for example L1, L2, or L3 cache or system memory. As such, the memory may include static random access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

1. A method of generating embeddings for a machine learning model, comprising:
extracting a character embedding and a word embedding from a first textual data;
generating a domain knowledge embedding from a domain knowledge dataset;
combining the character embedding, the word embedding, and the domain knowledge embedding into a combined embedding; and
providing the combined embedding to a layer of the machine learning model.

2. The method of claim 1, wherein the domain knowledge dataset includes feedback from a domain expert.

3. The method of claim 2, wherein the feedback from the domain expert includes named entity recognition labeling of a second textual data.

4. The method of claim 2, wherein the feedback from the domain expert includes additional vocabulary to be used to update a vocabulary database.

5. The method of claim 2, wherein the feedback from the domain expert is based upon a determination of the correctness of the output of the machine learning model.

6. The method of claim 1, wherein the domain knowledge dataset includes the output of a natural language processing engine applied to a second textual data.

7. The method of claim 1, wherein the domain knowledge dataset includes the output of a query based upon a second textual data to a TRIE dictionary based upon a vocabulary data.

8. The method of claim 1, wherein the machine learning model performs named entity recognition of a second textual data.

9. The method of claim 1, wherein the machine learning model performs medical disorder annotation of a second textual data.

10. The method of claim 1, further comprising:
training the machine learning model using the first textual data, the character embedding, and the word embedding before generating the domain knowledge embedding; and
retraining the machine learning model after generating the domain knowledge embedding.

11. The method of claim 10, further comprising:
determining that retraining of the machine learning model is required based upon the amount of data added to the domain knowledge dataset before retraining the machine learning model.

12. The method of claim 1, wherein extracting the character embedding further comprises:
applying a convolutional neural network layer to words in the first textual data to produce a first character embedding portion;
applying a long short term memory neural network layer to words in the first textual data to produce a second character embedding portion; and
concatenating the first character embedding portion and the second character embedding portion to produce the character embedding.

13. The method of claim 1, wherein the machine learning model includes a long short term memory layer and a conditional random field layer and further comprises providing the domain knowledge embedding to the conditional random field layer.

14. The method of claim 13, further comprising:
training the machine learning model using the first textual data, the character embedding and the word embedding before generating the domain knowledge embedding; and
retraining the machine learning model after generating the domain knowledge embedding.

15. A non-transitory machine-readable storage medium encoded with instructions for generating embeddings for a machine learning model, comprising:
instructions for extracting a character embedding and a word embedding from a first textual data;
instructions for generating a domain knowledge embedding from a domain knowledge dataset;
instructions for combining the character embedding, the word embedding, and the domain knowledge embedding into a combined embedding; and
instructions for providing the combined embedding to a layer of the machine learning model.

16. The non-transitory machine-readable storage medium of claim 15, wherein the domain knowledge dataset includes feedback from a domain expert.

17. The non-transitory machine-readable storage medium of claim 16, wherein the feedback from the domain expert includes named entity recognition labeling of a second textual data.

18. The non-transitory machine-readable storage medium of claim 16, wherein the feedback from the domain expert includes additional vocabulary to be used to update a vocabulary database.

19-28. (canceled)

29. A non-transitory machine-readable storage medium encoded with instructions for generating embeddings for a disorder annotation machine learning model, comprising:
instructions for extracting a character embedding and a word embedding from a first textual data;
instructions for generating a lexicon embedding from a lexicon dataset;
instructions for generating an extra tagging embedding from an extra tagging dataset;
instructions for combining the character embedding, the word embedding, the lexicon embedding, and extra tagging embedding into a combined embedding; and
instructions for providing the combined embedding to a layer of the disorder annotation machine learning model.

30. The non-transitory machine-readable storage medium of claim 29, wherein the extra tagging dataset includes feedback from a domain expert.

31.-39. (canceled)
